(19) United States
(12) Patent Application Publication
(10) Pub. No.: US 2010/0185573 A1
(43) Pub. Date: Jul. 22, 2010

(54) Method and Apparatus for Diagnosing an Allergy of the Upper Respiratory Tract Using a Neural Network

(76) Inventor: Paul Eirian Williams, Vale of Glamorgan (GB)

Correspondence Address: KING & SCHICKLI, PLLC, 247 NORTH BROADWAY, LEXINGTON, KY, 40507, US

(21) Appl. No.: 12/668,481

(22) PCT Filed: Jul. 10, 2008
(86) PCT No.: PCT/GB2008/002383

§ 371 Date: Jan. 11, 2010

(30) Foreign Application Priority Data
Jul. 11, 2007 (GB) 0713402.6

(51) Int. Cl. G06N 3/08; A61B 5/00; G01N 33/566; G06F 19/00

(52) U.S. Cl. 706/16; 424/9.81; 436/501; 702/19; 706/25

(57) ABSTRACT

The invention relates to a method and means for performing a diagnosis of a medical condition and, in particular, an allergy associated with the upper respiratory tract, using an artificial neural network.

### FIELD OF THE INVENTION

This invention relates to a method and means, including parts thereof, for diagnosing a medical condition, in particular an allergy associated with the upper respiratory tract, using an artificial neural network (ANN). The invention involves obtaining information about a patient, based on asking the patient a series of selected questions and carrying out a number of selected tests, inputting this information into a neural network, and obtaining a preliminary diagnosis. The invention applies equally to adults and children.

### BACKGROUND OF THE INVENTION

Allergies currently affect approximately 34% of the general population (Linneberg 2000). Whilst at one extreme serious conditions such as anaphylaxis can be life threatening, most allergic disorders pose little risk of death. However, diseases such as rhinitis, eczema and urticaria cause distress and misery for millions of patents, often at times in their lives when they should be most active (Holgate and Broide 2003). Allergic diseases are a significant cause of morbidity in modern society, adversely affecting sleep, intellectual functioning and recreational activities; food allergy may lead to considerable anxieties for fear of inadvertently ingesting the offending allergen (Holgate 1999). Furthermore, allergic diseases exert a profoundly negative impact on occupational performance and have major public health costs.

Across the United Kingdom, waiting times for specialist allergy consultations following referral from primary care are long.

The rising prevalence of allergies and the associated demand for specialist services suggest that waiting times will inevitably lengthen over the course of the next decade. Given that there is currently an acute shortage of Immunologists and Allergists in the UK and worldwide, it seems unlikely that sufficient medical manpower will emerge in the foreseeable future to deal with this increasing demand.

Recent in-house research has centred on the role of the Allergy Nurse Practitioner in the diagnosis and management of allergic disease. Increasing use of the Nurse Practitioner in a diagnostic role would enable waiting times to be shortened and new patient referrals to be seen without the presence of the Consultant Clinical Immunologist. Whilst Nurse Practitioner-based diagnosis and management strategies should, in time, ameliorate the critical situation, a parallel increase in demand for allergy services will, without doubt, limit the positive effects on waiting times. There therefore remains a need to develop further innovative methods to facilitate access of patients to clinical diagnostic services.

However, as one would expect, it is extremely important that any new methods of diagnosis are accurate if they are to be adopted by the medical community at large. These methods must be able to replicate, it not exceed, the accuracy of an experienced Clinical Immunologist. This is a difficult task to achieve because a Clinical Immunologist uses information from a vast number of sources when reaching a diagnosis.

Typically, when diagnosing a condition, a medical practitioner will integrate information from several sources, such as a medical history, a physical examination, the results of clinical tests, and by asking the patient about his/her condition. The medical practitioner will use judgement based on experience and intuition, both when deciding what to look for and in analysing the information, in order to come to a particular diagnosis.

Thus, the process of diagnosis involves a combination of knowledge, intuition and experience that leads a medical practitioner to ask certain questions and carry out particular clinical tests, and the validity of the diagnosis is very dependent upon these factors.

Given the predictive and intuitive nature of medical diagnosis, and the fact that specialist, experienced medical practitioners are in demand, we have attempted to replicate the diagnostic process in an automated system, in order to give a wider audience access to this service. We have found that artificial neural networks (ANNs) have characteristics that make them particularly well suited for this purpose.

ANNs are computational mathematical modelling tools for information processing and may be defined as ‘structures comprised of densely interconnected adaptive processing elements (nodes) that are capable of performing massively parallel computations for data processing and knowledge representation’ (Hecht-Nielsen 1990; Schalkoff 1977). Single artificial neurons for the computation of arithmetic and logical functions were first described by McCulloh and Pitts (1943); fifteen years later Rosenblatt (1958) described the first successful neurocomputer (the Mark 1 Perceptron). This simple network consisted of two layers of neurons connected by a single layer of weighted links and was capable of solving problems in a way analogous to information processing in the human brain (Wei et al 1998; Basheer and Hajmeer 2000).

These early structures were however unable to predict generalised solutions for complex non-linear problems. Over the course of the following five decades complexity has increased with the development of multiple networked perceptrons; such advances have led to the application of ANNs to a colossal number of problems, and by 1994 more than 50 different types of network were in existence (Pham 1994 and Basheer and Hajmeer 2000), each possessing unique properties enabling them to solve particular tasks.

Such ANNs are capable of dealing with non-linear data, fault and failure, high parallelism and imprecise and fuzzy information (Wei et al 1998). Neural networks have been shown to be capable of modelling complex real-world problems and found extensive acceptance in many scientific disciplines (Callan 1999). The decision as to which type of ANN should be utilised for a particular task depends on problem logistics, input type, and the execution speed of the trained network (Basheer and Hajmeer 2000).

Neural networks have found increasing application in a range of clinical settings where they have produced accurate and generalised solutions compared to traditional statistical methodology (reviewed Baxt 1995, Wei et al 1998, Dybowski and Gant 2001). For example, U.S. Pat. No. 6,678,669 discloses using an ANN to diagnose endometriosis, predicting pregnancy related events, such as the likelihood of delivery within a particular time period, and other such disorders relevant to women's health.

The most commonly used ANN in such studies is the Backpropagational Multilayer Perceptron (MLP). MLPs are particularly useful in solving pattern classification problems (Wei et al 1998; Basheer and Hajmeer, 2000), which are common in the clinical arena. In this context the ANN looks for patterns in a similar way to learning in the human mind; the more a particular pattern is represented, the stronger the recognition of it by the network.

Given the noisy, non-linear nature of clinical data utilised in the diagnosis of allergy, it has come to our attention that ANNs are a potential tool with which to facilitate access of patients to clinical diagnostic services, based on the hypothesis that ANNs can provide diagnosis for patients equivalent to that of the relevant specialists in the field.

To the best of our knowledge, this is the first time an ANN has been used to aid in the diagnosis of an allergy.

Accordingly, we have developed a method of diagnosing a medical condition using a neural network. In particular, from the vast amount of information that a clinician would have available, we have identified a manageable set of questions and tests that have clinical significance, and can be used to train a neural network to diagnose a condition, and by inputting the results of these questions and tests into a neural network thus trained the network to produce a diagnosis.

Surprisingly, we have found that an accurate diagnosis can be made by asking a patient just 5 questions and carrying out 4 medical tests, giving a total of 9 clinically significant inputs (referred to as the 9-input model), where it is currently standard practice for a medical practitioner to ask a patient up to 189 questions and carry out up to 21 different tests.

We have also identified a set of 12 (7 questions and 5 tests), 14 (7 questions and 7 tests), 15 (8 questions and 7 tests) 19 (11 questions and 8 tests), 21 (13 questions and 8 tests), 23 (14 questions and 8 tests) or 47 (29 questions and 18 tests) inputs, referred to in this description as the 12, 14, 15, 19, 21, 23 or 47-input models respectively, that can be input into a neural network to obtain a diagnosis.

The identification of these clinically significant questions and tests will mean that a neural network can be trained to diagnose a condition in considerably less time than it currently takes a consultant, which in turn will save time and money.

Additionally, a neural network offers an easy-to-use means of diagnosis, both for clinicians and non-clinicians, and will allow central aspects of diagnosis and management to be performed electronically in a way that is accessible to systematic audit and reduce inequalities in accessing allergy services, via the use of remote electronic information transfer.

According to a first aspect of the invention, there is therefore provided a method for diagnosing a condition comprising:

(a) asking a patient each of the following questions:
are any drugs being taken that are known to activate MAST cells (these drugs include alpha blockers, ACE inhibitors/ATII Receptor antagonists, aspirin, 5 HT-1 agonists, opiate or derivative medications, proton pump inhibitors, selective serotonin reuptake inhibitors and statins among others);
severity of nasal symptoms (on a scale of 0-n);
are the symptoms perennial/worse in the winter months;
are the symptoms worse during dusting and/or vacuuming/cleaning;
are the symptoms present after dietary salicylates; and

(b) carrying out each of the following tests:
skin prick test to house dust mite and/or cockroach;
skin prick test to mixed pollens;
RAST test result to house dust mite;
RAST test result to mixed pollens; and

(c) inputting the results of the questions and tests into a neural network that has been trained to diagnose said condition; and

(d) producing an output indicative of a diagnosis.

This is referred to as the 9-input model.

Reference herein to nasal symptoms includes any one or more of the following: nasal itching, sneezing runny nose, blocked nose, post-nasal drip, or itching of the palate

In a preferred method of the invention the results of the tests under part (b) above may be provided, as conventionally is the case, with a graded result and so represents an incremental unit indicative of the nature of the response. Alternatively, as is becoming increasingly popular, the results may represent a measure of a unit from a continuous scale such as kilo units of allergen-specific IgE antibodies per litre.

In a yet further preferred method of the invention said mixed pollens are selected having regard to the geographical region in which the patient lives. For example, in the UK, one would test for mixed grass pollens whereas in North America one is much more likely to include ragweed and in Northern Europe one is much more likely to test for tree birch. As will be apparent to the man skilled in the art the geographically representative allergens are well known in each geographical region and would be selected on the basis that in each region the selected allergens are known to elicit an allergic reaction of the upper respiratory tract.

The RAST test is undertaken using an antibody that is labelled with a suitable label such as a radio-label, although light emitting labels may be used as an alternative, and conventional techniques are used in order to measure the patient's immune status. RAST tests, and variations thereof, are well known to those skilled in the art and indeed have been performed for many decades. The original disclosure concerning diagnosis of an allergy by an in vitro test for allergen antibodies was described by Wide et al in 1967 and has further been assessed by Thomson & Bird, 1983.

In yet a further preferred method of the invention part (a) thereof further involves asking a patient each of the following questions:
are the symptoms worse indoors;
are the symptoms worse when gardening; and

part (b) thereof further includes carrying out the following further test:
RAST test result to cat.

This is referred to as a 12-input model.

In yet a further preferred method of the invention part (a) of the 12-input model includes asking a patient the following question:
severity of eye symptoms (on a scale of 0-n), instead of, are the symptoms worse indoors;

and part (b) of 12-input model further includes carrying out the following tests:
skin prick test result to cat; and
total IgE concentration.

This is referred to as the 14-input model.

Reference herein to eye symptoms includes reference to any one of the following: watery eyes, itchy eyes, red eyes, or gritty eyes.

According to further aspects and embodiments of the invention there are provided additional or alternative methodologies involving various additional inputs known as the 15-input, 19-input, 21-input, 23-input and 47-input models. The inputs comprise a series of questions and a series of tests. The questions are clearly indicated in Table 8 where an asterisk below the designator (reading from left to right 47, 23, 21, 19, 15, 14, 12, 9) for each input model is aligned with one of a series of questions, numbered 1-26, 45-47. Similarly, the tests are indicated by an asterisk below an input designator that is aligned with one of a series of tests, numbered 27-44. So, for example, the 15-input model involves asking questions 2, 5, 7, 13, 17, 24, 25 and 45 and also performing tests 27, 28, 30, 37, 38, 39 and 41.

The 19-input model involves asking questions 3, 5, 6, 7, 13, 17, 22, 24, 25, 26 and 45 and also performing tasks 27, 28, 30, 37, 38, 39, 41 and 42.

The 21-input model involves asking questions 2, 3, 5, 6, 7, 13, 17, 22, 24, 25, 26, 45 and 47 and also performing tests 27, 28, 30, 37, 38, 39, 41 and 42.

The 23-input model involves asking questions 2, 3, 5, 6, 7, 17, 18, 20, 22, 24, 25, 26, 45 and 47 and also performing tests 27, 28, 30, 37, 38, 39, 41 and 42.

The 47-input model involves asking questions 1-26, 45-47 and also performing tests 27-44.

As mentioned, in a preferred method of the invention the results of the tests under part (b) above may be provided, as conventionally is the case, with a graded result and so represents an incremental unit indicative of the nature of the response. Alternatively, as is becoming increasingly popular, the results may represent a measure of a unit from a continuous scale such as kilo units of allergen-specific IgE antibodies per litre.

Further, as mentioned said mixed grass or tree pollen may be substituted for a pollen that is representative of the geographical region in which the patient lives. For example, in the UK, one would test for mixed grass pollens whereas in North America one is much more likely to test for ragweed and in Northern Europe, one is much more likely to test for tree birch. As will be apparent to the man skilled in the art the geographically representative pollen is well known in each geographical region and would be selected on the basis that in each region the selected pollen is known to elicit an allergic reaction of the upper respiratory tract.

In some cases it may be useful to save results for analysis at a later time, for example if they cannot be obtained simultaneously. In this instance the results may be stored on a computer system and applied to a neural network subsequently.

In another aspect of the invention, there is provided a computer system or apparatus, configured to aid in the diagnosis of a condition, comprising:
(a) a device for obtaining data relating to a patient, wherein the data comprises answers to any selected combination of questions and results of tests outlined in any of the 9-, 12-, 14-, 15-, 19-, 21-, 23- or 47-input models outlined above;
(b) optionally, a device for storing the data in storage means of the computer system;
(c) a device for transferring the data to a neural network trained on samples of the data; and
(d) a device for extracting from the trained neural network an output, the output being an indicator for the diagnosis of the condition.

In a preferred computer system or apparatus the data comprises information obtained using the 9-, 12-, 14-, 15-, 19-, 21-, 23- or 47-input model.

As will be appreciated, this aspect of the invention may also be adapted so that the computer is linked to an intranet or Internet with a neural network, thereby allowing patients and/or medical practitioners to input information from remote locations and obtain a preliminary diagnosis.

The results of any of the 9-, 12-, 14-, 15-, 19-, 21-, 23- and 47-input models, or any selected combination thereof, may also be used to train a neural network to diagnose a condition.

Accordingly, in a further aspect of the invention there is provided a method for training a neural network to aid in diagnosing a condition, comprising:
a) obtaining data relating to a group of patients in whom the condition is known, wherein the data comprises any selected combination of the results of the questions and tests outlined in any of the 9-, 12-, 14-, 15-, 19-, 21-, 23- or 47-input models;
(b) training a neural network to identify the pattern of data which corresponds to the condition; and
(c) storing the neural network in storage means of a computer or on a computer-readable medium.

A neural network may also be trained using other methods, which methods will be apparent to a man skilled in the art.

The invention further comprises a computer or a computer system comprising at least one neural network embodying any one or more of the aforementioned models or methods for the purposes of performing a diagnosis.

Furthermore, the invention comprises at least one neural network that has been trained for diagnosis using data from the 9-, 12-, 14-, 15-, 19-, 21-, 23- or 47-input models. Such a neural network may be sold separately, or put on a server so that it can be accessed remotely.

Yet further, the invention comprises a data carrier comprising the aforementioned methodology of the invention and/or a software interface for enabling a user to communicate with a neural network trained for the diagnostic purpose of the invention.

According to another aspect of the present invention there is provided a computer program product comprising:

a computer usable medium having computer readable program code and computer readable system code embodied on said medium for aiding in the diagnosis of a condition, said computer program product including:

computer program code means, when the program code is loaded, to make the computer execute a procedure to:
(a) obtain data relating to a patient, wherein the data comprises answers to any selected combination of questions and results of the tests outlined in any of 9-, 12-, 14-, 15-, 19-, 21-, 23- or 47-input models above;
(b) optionally, store the data;
(c) transfer the data to a neural network trained on the aforementioned data; and
(d) extract from the trained neural network an output, the output being an indicator for the diagnosis of the condition.

According to a further aspect of the invention there is provided a computer system comprising a first means for:
(a) obtaining data relating to a patient, wherein the data comprises answers to any selected combination of questions and results of tests outlined in any of 9-, 12-, 14-, 15-, 19-, 21-, 23- or 47-input models above; and

a second remote means, wherein said second means comprises means for:
(b) optionally, storing the data;
(c) transferring the data to a neural network trained on the aforementioned data; and
(d) extracting from the trained neural network on output, the output being an indicator for the diagnosis of the condition.

In one embodiment of the invention, the condition to be diagnosed is an allergy associated with the upper respiratory tract. The term ‘allergy’ in this context is taken to mean any disease, condition or disorder in which the immune system is triggered by a substance to which it has become sensitive.

In another embodiment, the condition to be diagnosed is rhinitis or sinusitis. ‘Rhinitis’ is taken to mean any condition that results in the inflammation of the nasal mucous membrane, and includes conditions such as allergic perennial rhinitis, allergic seasonal rhinitis, idiopathic perennial rhinitis, idiopathic seasonal rhinitis, drug-induced rhinitis, dietary salicylate-induced rhinitis, rhino-sinusitis or rhino-conjunctivitis. ‘Sinusitis’ is taken to mean a condition resulting in inflammation of any one of the air-containing cavities of the skull that communicate with the nose, and includes conditions such as ethmoid sinusitis, frontal sinusitis, maxillary sinusitis, sphenoid sinusitis and nasal sinusitis.

In yet another embodiment of the invention, the condition to be diagnosed is any one of the following:
allergic perennial rhinitis, allergic seasonal rhinitis, idiopathic perennial rhinitis, idiopathic seasonal rhinitis, drug-induced rhinitis, dietary salicylate-induced rhinitis or rhino-sinusitis.

The present invention will now be illustrated with reference to the following method and results.

### EXAMPLE 1

Table 1 shows the distribution of diagnoses in patients presenting to the Welsh Clinical Allergy Service outpatient clinics in 2001, and is representative of the caseload seen in this regional allergy centre. Given the high proportion of patients presenting to the service with symptoms of rhinitis, it was decided to utilise this patient group for our study.

TABLE 1
Distribution of diagnoses in patients seen
in WCAS outpatient clinics in 2001 (n = 213)

| | No. of patients | Percentage of |
|---|---|---|
| Diagnostic Category | with diagnosis | all patients (%) |
| Urticaria/Angioedema | 46 | 21.6 |
| Rhinitis | 43 | 20.2 |
| Drug-induced angioedema/reaction | 28 | 13.1 |
| Food allergy | 26 | 12.2 |
| Food intolerance | 14 | 6.6 |
| Salicylate intolerance | 11 | 5.2 |
| Venom insensitivity | 7 | 3.3 |
| Non-allergic/miscellaneous conditions | 38 | 17.8 |
| Total | 213 | 100 |

### Methods

### Ethical Considerations

Bro Taf Local Research Ethics Committee granted ethical approval for all aspects of this study and the project was registered with Cardiff and Vale NHS Trust Research and Development Office. All participants were required to complete a consent form. Data was anonymised prior to analysis and handled in accordance with the Data Protection Act 1998.

### Structured Questionnaire Design

This study made use of a standard questionnaire (Table 7) comprising 189 questions and 6 tests were created using the commercial Cardiff TELEform information capture system v7.0 Designer module. This questionnaire was devised as an integral part of the Nurse Practitioner-based diagnosis and management evaluation program and aimed to gather demographic and clinical information in a structured format. This questionnaire was endorsed by a multidisciplinary panel of experts and piloted in WCAS clinics throughout 2001.

### Patient Recruitment and Data Collection

Patients aged 18 to 75 referred to the WCAS by General Practitioners or hospital doctors due to symptoms of rhinitis were drawn from the routine non-urgent outpatient waiting list and recruited using an approved protocol. All consenting patients with predominant presenting symptoms of rhinitis were entered into the study. There were no exclusion criteria. Participants underwent Skin Prick Testing immediately prior to an initial conventional consultation with either the Consultant Clinical Immunologist or Allergy Nurse Practitioner. The order of consultation was randomized so that roughly equal numbers of patients were seen first by the Nurse Practitioner as by the Consultation Clinical Immunologist. Findings were recorded on the standard questionnaire ensuring all sections were fully completed. Patients were then seen independently by the other practitioner, and findings annotated upon a separate questionnaire. Total serum IgE and RAST testing were performed upon clinical discretion. As per current WCAS protocol, a clinic letter outlining the final diagnosis and management plan was dictated by the Consultant Clinical Immunologist and posted to the referring medical practitioner and patient. A similar letter was dictated independently by the Allergy Nurse Practitioner, which was retained as supporting evidence to her questionnaire, for analysis in a later study.

### Data Transfer

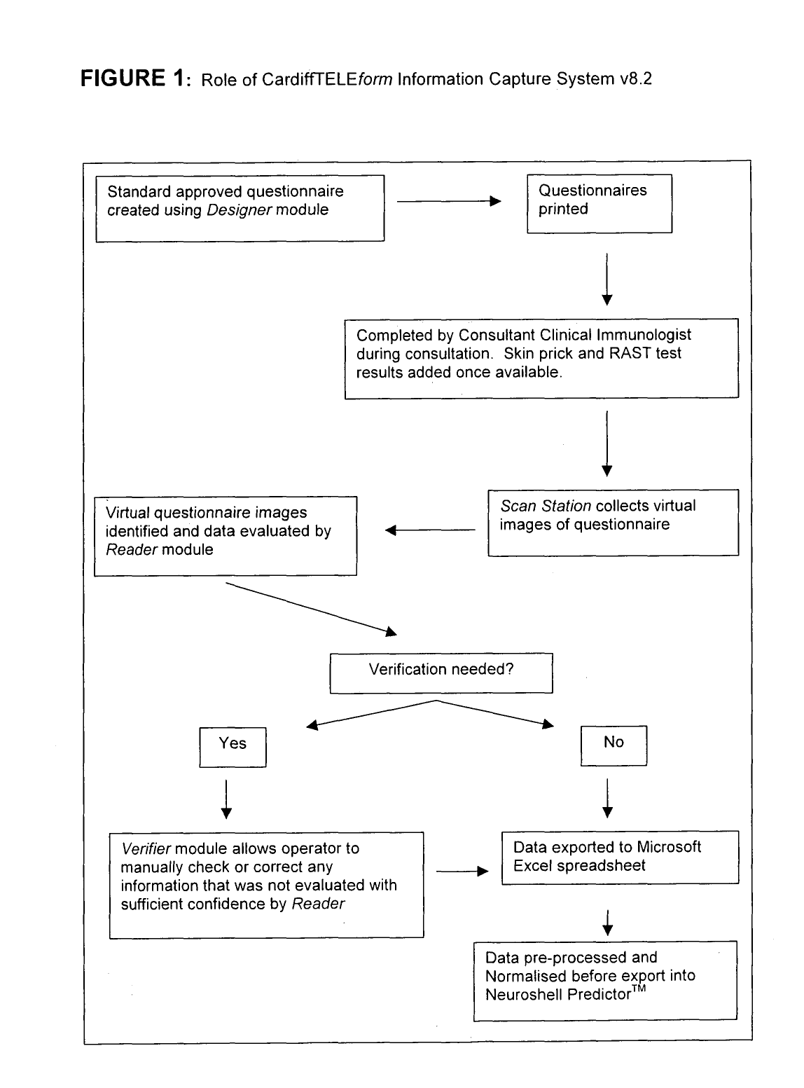

Once available, all RAST and other test results were added to data recorded during respective consultations. Completed questionnaires were processed using the commercial Cardiff TELEform information capture system v8.2 Scan station, Reader and Verifier modules (see FIG. 1)./Data was exported into separate Microsoft Excel files for each clinician.

### Data Preprocessing and Normalisation

Data imported into Microsoft Excel was anonymised. All input variables were inspected for transfer accuracy and errors corrected manually. Data was normalised (scaled) within a uniform range for each input variable, some variables removed (e.g. domestic demographic data, ethnic origin and marital status) and a number of new input variables created following recoding of defined input groups (e.g. 17 inputs assessing the presence of asthma, eczema, hayfever or perennial rhinitis in the patients mother, father, siblings or children recoded as a single input—‘positive family history’). The final aetiological diagnosis for each patient was coded into one of six output categories (allergic perennial, allergic seasonal, idiopathic perennial, idiopathic seasonal, drug induced or dietary salicylate-induced rhinitis).

### Data Partitioning

Data was partitioned into two separate Excel parent databases (i.e. separate Excel worksheets) (i) ‘all questionnaire inputs’ (189 input variables; six output variables) and (ii) ‘clinically selected inputs’ (47 input variables; six output variables) (see Tables 7 and 8), as it became available. ANN models were developed using data and diagnoses from the Consultant Clinical Immunologist. Model development required data from each parent database to be divided into two subsets: (i) training and test data and (ii) validation.

At present there are no mathematical rules governing the required size of data subsets and most ANN-based studies utilize anecdotal rules derived from experience and analogy with statistical regression techniques (Basheer and Hajmeer et al 2000). Data utilised for the ANN training and test subset for both parent databases was drawn from patients 001-062 since these were collected first and data from patients 063-093 were used as test data.

### Balancing of Training and Test Subset Data

It is desirable that data used in ANN training is nearly evenly distributed between output categories to prevent the ANN model generated from being biased to over-represented output classes (Swingler 1996). Table 2 shows the distribution of diagnoses amongst patients 001-062. Traditional approaches to dealing with such unbalanced data include removing examples from over-represented output classes or adding examples pertaining to under-represented classes (Basheer and. Hajmeer 2000). The relatively small size of the training and test data subset (62 patients) made the first option undesirable. Furthermore, whilst there is no published epidemiological data with which to compare the distribution of diagnoses in these first 62 patients, it seemed unlikely that significant numbers of under-represented diagnoses would be made in patients 063-093. It was therefore decided to use unbalanced training and test data on the premise that models created would reflect what appeared to be a real-world bias to allergic perennial rhinitis in patients presenting to the WCAS.

TABLE 2
Distribution of Diagnoses in Patients 001-
062 (ANN Training and test data subset)

| | No. of patients | Percentage of |
|---|---|---|
| Diagnostic Output Category | with diagnosis | all patients (%) |
| Allergic Perennial Rhinitis | 34 | 55 |
| Allergic Seasonal Rhinitis | 7 | 11 |
| Idiopathic Perennial Rhinitis | 13 | 21 |
| Idiopathic Seasonal Rhinitis | 1 | 2 |
| Drug-induced Rhinitis | 5 | 8 |
| Dietary salicylate-induced Rhinitis | 2 | 3 |
| Total | 62 | 100 |

### Optimisation of ANN Architecture

The study used a commercially available ANN the Neuroshell Predictor™. Neuroshell Predictor™ can operate in one of two modes: neural mode of analysis, this uses a neural net that dynamically grows hidden neurons to build a model which generalises well and trains quickly. When applying the trained network to new data, the Neural Training Strategy may enable better results to be obtained on “noisy data” that is somewhat dissimilar from the data used to train the network.

Alternatively, the Neuroshell Predictor™ can be used in a genetic mode of analysis. The Genetic Training Strategy trains slowly. When applying the trained network to new data, the Genetic Training Strategy gets better results when the new data is similar to the training data. It also works better when the training data is sparse.

### Neuroshell Predictor™ Data Output Format in Neural Analysis Mode

The Neuroshell Predictor™ analysis of 47 input fields in neural analysis mode is shown below. The program optimised the analysis of the data on patients 1-62 (training data), with an upper limit of hidden nodes of 100. The program calculated that 4 hidden nodes were optimal, and produced the Table below classifying the input data into different categories.

TABLE 3
Patients 1-62 Training data for neural learning

| | Actual | Actual | | | | | |
|---|---|---|---|---|---|---|---|
| | “Allergic | “Allergic | Actual | Actual | Actual | Actual | |
| | perennial | seasonal | “Drug | “Idiopathic | “Idiopathic | “Salicylate | |
| | (hdm)” | (mgp)” | induced” | perennial” | seasonal” | induced” | Total |
| Classified as | 34 | 0 | 0 | 0 | 0 | 0 | 34 |
| “Allergic | | | | | | | |
| perennial | | | | | | | |
| (hdm)” | | | | | | | |
| Classified as | 0 | 6 | 0 | 0 | 0 | 0 | 6 |
| “Allergic | | | | | | | |
| seasonal | | | | | | | |
| (mgp)” | | | | | | | |
| Classified as | 0 | 0 | 5 | 0 | 0 | 0 | 5 |
| “Drug | | | | | | | |
| induced” | | | | | | | |
| Classified as | 0 | 1 | 0 | 13 | 0 | 0 | 14 |
| “Idiopathic | | | | | | | |
| perennial” | | | | | | | |
| Classified as | 0 | 0 | 0 | 0 | 1 | 0 | 1 |
| “Idiopathic | | | | | | | |
| seasonal” | | | | | | | |
| Classified as | 0 | 0 | 0 | 0 | 0 | 2 | 2 |
| “Salicylate | | | | | | | |
| induced” | | | | | | | |
| Total | 34 | 7 | 5 | 13 | 1 | 2 | 62 |
| True-pos. | 1 | 0.8571 | 1 | 1 | 1 | 1 | |
| ratio | | | | | | | |
| False-pos. | 0 | 0 | 0 | 0.0204 | 0 | 0 | |
| ratio | | | | | | | |
| True-neg. | 1 | 1 | 1 | 0.9796 | 1 | 1 | |
| ratio | | | | | | | |
| False-neg. | 0 | 0.1429 | 0 | 0 | 0 | 0 | |
| ratio | | | | | | | |
| Sensitivity | 100.00% | 85.71% | 100.00% | 100.00% | 100.00% | 100.00% | |
| Specificity | 100.00% | 100.00% | 100.00% | 97.96% | 100.00% | 100.00% | |

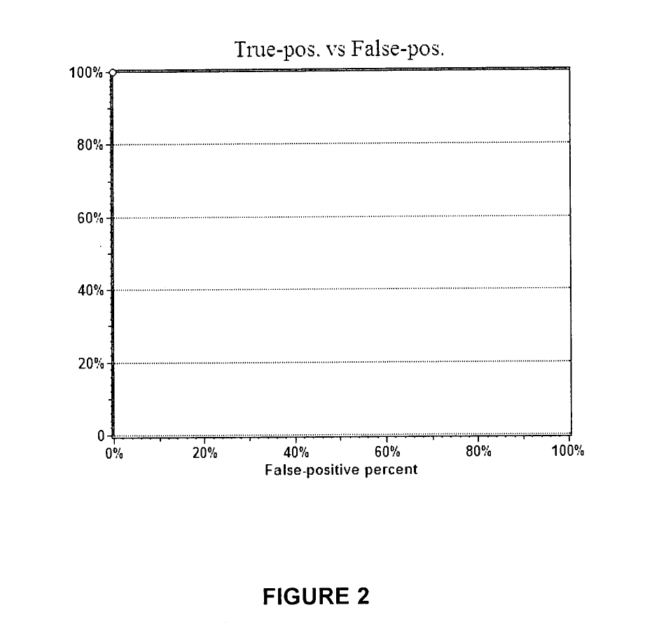

In the above Table row 9, designated as Total, indicates the number of patients that were clinically diagnosed as having the condition described at the top of each column. For example, in the second column a clinical diagnosis indicated that 34 of the patients (from Group 1-62) had allergic perennial rhinitis to house dust mite. Using data from these patients the Neuroshell Predictor™ programme was trained so that it too classified the same 34 patients as having allergic perennial rhinitis to house dust mites. In other words there was 100% match. This was true of all the other columns except for column 3 labelled “allergic seasonal mixed grass pollen” where, of the 7 individuals (from Groups 1-62) that were clinically diagnosed as having allergic seasonal mixed grass pollen rhinitis, 6 were classified as such by the ANN program whereas one was classified as having idiopathic perennial rhinitis. In other words there was not quite a 100% match. Nevertheless, having regard to FIG. 2 it can be seen from the ROC curve that there was almost a 100% match. It can therefore be seen that training the ANN program when in neural mode of analysis worked extremely well. Accordingly, when the trained ANN operating in this mode was given test data, i.e. data from patients 63-92, the results in Table 4 were obtained.

TABLE 4
Patients 63-92 Training data for neural learning

| | Actual | Actual | | | | | |
|---|---|---|---|---|---|---|---|
| | “Allergic | “Allergic | Actual | Actual | Actual | Actual | |
| | perennial | seasonal | “Drug | “Idiopathic | “Idiopathic | “Salicylate | |
| | (hdm)” | (mgp)” | induced” | perennial” | seasonal” | induced” | Total |
| Classified as | 12 | 1 | 0 | 0 | 0 | 1 | 14 |
| “Allergic | | | | | | | |
| perennial | | | | | | | |
| (hdm)” | | | | | | | |
| Classified as | 1 | 2 | 0 | 0 | 0 | 0 | 3 |
| “Allergic | | | | | | | |
| seasonal | | | | | | | |
| (mgp)” | | | | | | | |
| Classified as | 2 | 2 | 0 | 0 | 0 | 0 | 4 |
| “Drug | | | | | | | |
| induced” | | | | | | | |
| Classified as | 1 | 2 | 1 | 4 | 0 | 1 | 9 |
| “Idiopathic | | | | | | | |
| perennial” | | | | | | | |
| Classified as | 0 | 0 | 0 | 0 | 0 | 0 | 0 |
| “Idiopathic | | | | | | | |
| seasonal” | | | | | | | |
| Classified as | 0 | 0 | 0 | 0 | 0 | 0 | 0 |
| “Salicylate | | | | | | | |
| induced” | | | | | | | |
| Total | 16 | 7 | 1 | 4 | 0 | 2 | 30 |
| True-pos. | 0.75 | 0.2857 | 0 | 1 | N/A | 0 | |
| ratio | | | | | | | |
| False-pos. | 0.1429 | 0.0435 | 0.1379 | 0.1923 | 0 | 0 | |
| ratio | | | | | | | |
| True-neg. | 0.8571 | 0.9565 | 0.8621 | 0.8077 | 1 | 1 | |
| ratio | | | | | | | |
| False-neg. | 0.25 | 0.7143 | 1 | 0 | 0 | 1 | |
| ratio | | | | | | | |
| Sensitivity | 75.00% | 28.57% | 0.00% | 100.00% | 0.00% | 0.00% | |
| Specificity | 85.71% | 95.65% | 86.21% | 80.77% | 100.00% | 100.00% | |

It can therefore been seen that of the 16 patients (from Group 63-92) that were classified by a clinician as suffering from allergic perennial house dust mite rhinitis, 12 of these individuals were classified by the ANN program as suffering from the same condition. One further individual was classified by the ANN as suffering from allergic seasonal mixed grain pollen rhinitis, 2 were classified as suffering from drug induced rhinitis and one was classified as suffering from idiopathic perennial rhinitis.

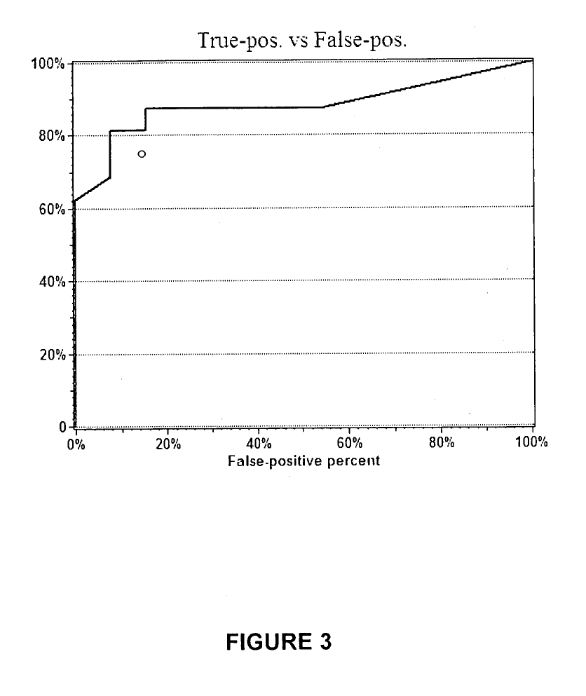

The ROC curve for this data is shown in FIG. 3 where it can be seen that there is a satisfactory correlation.

### Neuroshell Predictor™ Data Output Format in Genetic Analysis Mode

When the Neuroshell Predictor™ program was run in the genetic mode of analysis the data shown in Table 5 was obtained.

TABLE 5
Patients 1-62 Training data for genetic learning

| | Actual | Actual | | | | | |
|---|---|---|---|---|---|---|---|
| | “Allergic | “Allergic | Actual | Actual | Actual | Actual | |
| | perennial | seasonal | “Drug | “Idiopathic | “Idiopathic | “Salicylate | |
| | (hdm)” | (mgp)” | induced” | perennial” | seasonal” | induced” | Total |
| Classified as | 33 | 1 | 0 | 0 | 0 | 0 | 34 |
| “Allergic | | | | | | | |
| perennial | | | | | | | |
| (hdm)” | | | | | | | |
| Classified as | 1 | 5 | 0 | 0 | 0 | 0 | 6 |
| “Allergic | | | | | | | |
| seasonal | | | | | | | |
| (mgp)” | | | | | | | |
| Classified as | 0 | 0 | 5 | 1 | 0 | 0 | 6 |
| “Drug | | | | | | | |
| induced” | | | | | | | |
| Classified as | 0 | 1 | 0 | 12 | 1 | 0 | 14 |
| “Idiopathic | | | | | | | |
| perennial” | | | | | | | |
| Classified as | 0 | 0 | 0 | 0 | 0 | 0 | 0 |
| “Idiopathic | | | | | | | |
| seasonal” | | | | | | | |
| Classified as | 0 | 0 | 0 | 0 | 0 | 2 | 2 |
| “Salicylate | | | | | | | |
| induced” | | | | | | | |
| Total | 34 | 7 | 5 | 13 | 1 | 2 | 62 |
| True-pos. | 0.9706 | 0.7143 | 1 | 0.9231 | 0 | 1 | |
| ratio | | | | | | | |
| False-pos. | 0.0357 | 0.0182 | 0.0175 | 0.0408 | 0 | 0 | |
| ratio | | | | | | | |
| True-neg. | 0.9643 | 0.9818 | 0.9825 | 0.9592 | 1 | 1 | |
| ratio | | | | | | | |
| False-neg. | 0.0294 | 0.2857 | 0 | 0.0769 | 1 | 0 | |
| ratio | | | | | | | |
| Sensitivity | 97.06% | 71.43% | 100.00% | 92.31% | 0.00% | 100.00% | |
| Specificity | 96.43% | 98.18% | 98.25% | 95.92% | 100.00% | 100.00% | |

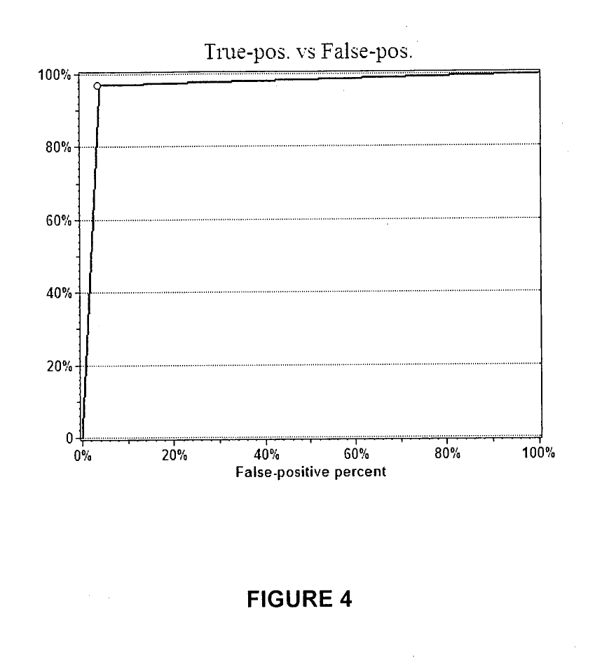

Here it can be seen that there is an extremely good correlation for diagnosing allergic perennial house dust mite rhinitis and allergic seasonal mixed grain pollen rhinitis. In the former instance, of the 34 individuals (from patients 1-62) that were classified by a clinician as suffering from allergic perennial house dust mite rhinitis, 33 were also similarly classified by the ANN. In the latter instance, of the 7 individuals (from Group 1-62) that were diagnosed by a clinician as suffering from allergic seasonal mixed grain pollen rhinitis 5 were similarly classified by the ANN. One individual was further classified as suffering from allergic perennial rhinitis and a further was classified as suffering from idiopathic perennial rhinitis. The ROC curve for this data is shown in FIG. 4.

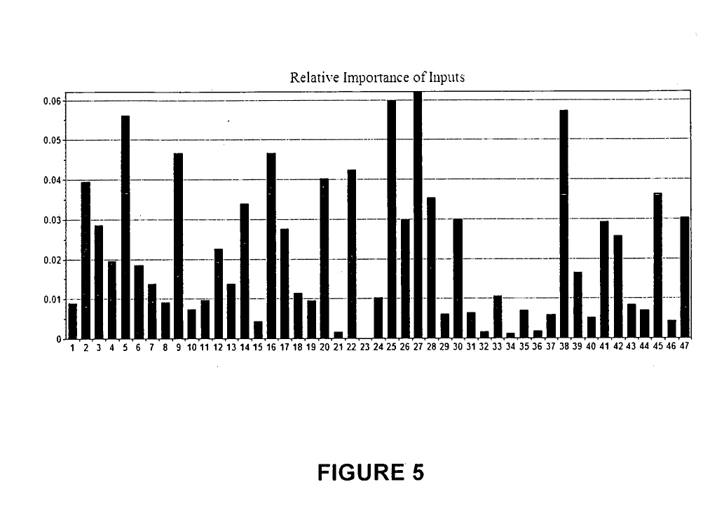

The relative importance of all the data entries as assessed and of the 189 questions shown in Table 7, 47 were considered to be particularly important. These 47 questions are shown in Table 8. Moreover, the ANN software program produced a graph showing the relative importance of these selected 47 questions and this data is indicated in FIG. 5.

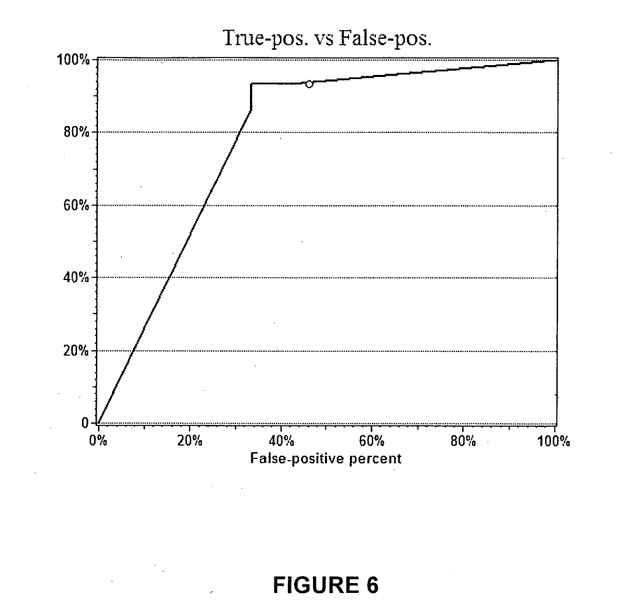

Once the Neuroshell Predictor™ program had been trained in the genetic mode of analysis test data from patients 63-92 was fed therein and the data shown in Table 6 was obtained. The ROC curve for this data is shown in FIG. 6.

TABLE 6
Patients 63-92 Training data for genetic learning

| | Actual | Actual | | | | | |
|---|---|---|---|---|---|---|---|
| | “Allergic | “Allergic | Actual | Actual | Actual | Actual | |
| | perennial | seasonal | “Drug | “Idiopathic | “Idiopathic | “Salicylate | |
| | (hdm)” | (mgp)” | induced” | perennial” | seasonal” | induced” | Total |
| Classified as | 14 | 5 | 0 | 0 | 0 | 1 | 20 |
| “Allergic | | | | | | | |
| perennial | | | | | | | |
| (hdm)” | | | | | | | |
| Classified as | 0 | 1 | 0 | 2 | 0 | 0 | 3 |
| “Allergic | | | | | | | |
| seasonal | | | | | | | |
| (mgp)” | | | | | | | |
| Classified as | 0 | 0 | 1 | 0 | 0 | 0 | 1 |
| “Drug | | | | | | | |
| induced” | | | | | | | |
| Classified as | 1 | 1 | 0 | 2 | 0 | 0 | 4 |
| “Idiopathic | | | | | | | |
| perennial” | | | | | | | |
| Classified as | 0 | 0 | 0 | 0 | 0 | 0 | 0 |
| “Idiopathic | | | | | | | |
| seasonal” | | | | | | | |
| Classified as | 0 | 0 | 0 | 0 | 0 | 0 | 0 |
| “Salicylate | | | | | | | |
| induced” | | | | | | | |
| Total | 15 | 7 | 1 | 4 | 0 | 1 | 28 |
| True-pos. | 0.9333 | 0.1429 | 1 | 0.5 | N/A | 0 | |
| ratio | | | | | | | |
| False-pos. | 0.4615 | 0.0952 | 0 | 0.0833 | 0 | 0 | |
| ratio | | | | | | | |
| True-neg. | 0.5385 | 0.9048 | 1 | 0.9167 | 1 | 1 | |
| ratio | | | | | | | |
| False-neg. | 0.0667 | 0.8571 | 0 | 0.5 | 0 | 1 | |
| ratio | | | | | | | |
| Sensitivity | 93.33% | 14.29% | 100.00% | 50.00% | 0.00% | 0.00% | |
| Specificity | 53.85% | 90.48% | 100.00% | 91.67% | 100.00% | 100.00% | |

Data Analysis with View to Optimising Data Input and Diagnosis

The information shown in Tables 3-6 and FIGS. 1-6 clearly show that the commercially available product Neuroshell Predictor™ can be used to produce an ANN that is capable of performing a clinical diagnosis. However, further data analysis is needed in order to determine the optimum number of reliable data inputs needed to obtain an acceptable tool for diagnosis. Accordingly, the number and combination of data inputs was progressively reduced and varied, respectively, with a view to determining a preferred number and nature of inputs for producing a reliable diagnosis. In Table 8 we present the results of eight input models using 47, 23, 21, 19, 15, 14, 12 or 9 data inputs. The inputs are specified having regard to indicators 1-47 which represent one of a number of questions or tests listed in column 1 of Table 8. Using each input model, and each mode of operation of the ANN, data was obtained concerning the ANN reliability of diagnosis vis a vis use of clinical analysis.

TABLE 7
189 ‘Questionnaire’ Inputs and Normalised Values

| | | | | Models input | | | | |
|---|---|---|---|---|---|---|---|---|
| | | | Normalised | utilised in (*): | | | | |
| No | Input code | Input question | values | 189 | 40 | 20 | 15 | 10 |
| 1 | Sex | Patent gender | 1 = male | * | * | | | |
| | | | 2 = female | | | | | |
| 2 | Age | Patient age in years | — | * | | | | |
| 3 | DIY/Dec | Indoor hobbies: DIY/Decorating | 0 = no | * | | | | |
| | | | 1 = yes | | | | | |
| 4 | Cooking | Indoor hobbies: Cooking | 0 = no | * | * | | | |
| | | | 1 = yes | | | | | |
| 5 | Arts | Indoor hobbies: Arts/Crafts | 0 = no | * | | | | |
| | | | 1 = yes | | | | | |
| 6 | Gardening | Outdoor hobbies: Gardening | 0 = no | * | * | | | |
| | | | 1 = yes | | | | | |
| 7 | Sports | Outdoor hobbies: Sports | 0 = no | * | | | | |
| | | | 1 = yes | | | | | |
| 8 | Walking | Outdoor hobbies: Walking | 0 = no | * | * | * | * | |
| | | | 1 = yes | | | | | |
| 9 | NumHosAdmiss | Number of hospital in-patient admissions | — | * | | | | |
| 10 | Eczema | Suffer from eczema | 0 = no | * | | | | |
| | | | 1 = yes | | | | | |
| 11 | Asthma | Suffer from asthma | 0 = no | * | | | | |
| | | | 1 = yes | | | | | |
| 12 | High BP | Suffer from hypertension | 0 = no | * | * | * | * | |
| | | | 1 = yes | | | | | |
| 13 | Arthritis | Suffer from arthritis | 0 = no | * | | | | |
| | | | 1 = yes | | | | | |
| 14 | Thyroid | Suffer from thyroid trouble | 0 = no | * | * | * | | |
| | | | 1 = yes | | | | | |
| 15 | AlphaBlocker | Take any Alpha Blockers | 0 = no | * | | | | |
| | | | 1 = yes | | | | | |
| 16 | ACE/ATII | Take any ACE Inhibitors/ATII Receptor | 0 = no | * | * | * | * | * |
| | | antagonists | 1 = yes | | | | | |
| 17 | Aspirin | Take Aspirin | 0 = no | * | | | | |
| | | | 1 = yes | | | | | |
| 18 | FemHormonal | Take any products containing female sex | 0 = no | * | | | | |
| | | hormones | 1 = yes | | | | | |
| 19 | 5HT1Agonist | Take any 5HT1 Agonsts | 0 = no | * | | | | |
| | | | 1 = yes | | | | | |
| 20 | InhalB2Agonist | Take any inhaled B2 Agonists | 0 = no | * | | | | |
| | | | 1 = yes | | | | | |
| 21 | InhalCorticosteroid | Take any inhaled corticosteroids | 0 = no | * | | | | |
| | | | 1 = yes | | | | | |
| 22 | Opiates + derivatve | Take any opiate or derivative medications | 0 = no | * | | | | |
| | | | 1 = yes | | | | | |
| 23 | PPI | Take any Proton Pump Inhibitors | 0 = no | * | | | | |
| | | | 1 = yes | | | | | |
| 24 | SSRI | Take any Selective Serotonin Re-uptake | 0 = no | * | | | | |
| | | inhibitors | 1 = yes | | | | | |
| 25 | Statins | Take any Statins | 0 = no | * | | | | |
| | | | 1 = yes | | | | | |
| 26 | TCA | Take any Tricyclic Antidepressants | 0 = no | * | * | | | |
| | | | 1 = yes | | | | | |
| 27 | Smoker | Do you smoke | 0 = never | * | | | | |
| | | | 1 = previously | | | | | |
| | | | 2 = currently | | | | | |
| 28 | Cigarette | Smoke/smoked cigarettes | 0 = no | * | * | * | | |
| | | | 1 = yes | | | | | |
| 29 | Pipe | Smoked/smoked a pipe | 0 = no | * | | | | |
| | | | 1 = yes | | | | | |
| 30 | NumYrsSmoked | Number of years smoking | — | * | | | | |
| 31 | NumCigDay | Number of cigarettes smoked per day | — | * | | | | |
| 32 | MumAst | Mother suffers from asthma | 0 = no | * | | | | |
| | | | 1 = yes | | | | | |
| 33 | MumEcz | Mother suffers from eczema | 0 = no | * | * | | | |
| | | | 1 = yes | | | | | |
| 34 | MumHay | Mother suffers from hayfever | 0 = no | * | | | | |
| | | | 1 = yes | | | | | |
| 35 | MumRhin | Mother suffers from perennial rhinitis | 0 = no | * | | | | |
| | | | 1 = yes | | | | | |
| 36 | DadAst | Father suffers from asthma | 0 = no | * | | | | |
| | | | 1 = yes | | | | | |
| 37 | DadHay | Father suffers from hayfever | 0 = no | * | | | | |
| | | | 1 = yes | | | | | |
| 38 | BroAst | Brother(s) suffer from asthma | 0 = no | * | | | | |
| | | | 1 = yes | | | | | |
| 39 | BroEcz | Brother(s) suffer from eczema | 0 = no | * | | | | |
| | | | 1 = yes | | | | | |
| 40 | BroHay | Brother(s) suffer from hayfever | 0 = no | * | * | | | |
| | | | 1 = yes | | | | | |
| 41 | BroRhin | Brother(s) suffer from perennial rhinitis | 0 = no | * | | | | |
| | | | 1 = yes | | | | | |
| 42 | SisAst | Sister(s) suffer from asthma | 0 = no | * | | | | |
| | | | 1 = yes | | | | | |
| 43 | SisEcz | Sister(s) surf from eczema | 0 = no | * | | | | |
| | | | 1 = yes | | | | | |
| 44 | SisHay | Sister(s) suffer from hayfever | 0 = no | * | | | | |
| | | | 1 = yes | | | | | |
| 45 | ChildAst | Children suffer from asthma | 0 = no | * | | | | |
| | | | 1 = yes | | | | | |
| 46 | ChildEcz | Children suffer from eczema | 0 = no | * | | | | |
| | | | 1 = yes | | | | | |
| 47 | ChildHay | Children suffer from hayfever | 0 = no | * | | | | |
| | | | 1 = yes | | | | | |
| 48 | ChildRhin | Children suffer from rhinitis | 0 = no | * | | | | |
| | | | 1 = yes | | | | | |
| 49 | SevRunNose | Severity of runny nose | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 50 | SevitchNose | Severity of itchy nose | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 51 | SevBlkNose | Severity of blocked nose | 0 = no symptoms | * | * | | | |
| | | | 1 = very mild | | | | | |
| | | | 2 = mild | | | | | |
| | | | 3 = moderate | | | | | |
| | | | 4 = severe | | | | | |
| 52 | SevPostNasDisch | Severity of post nasal discharge | 0 = no symptoms | | | | | |
| | | | 1 = very mild | | | | | |
| | | | 2 = mild | | | | | |
| | | | 3 = moderate | | | | | |
| | | | 4 = severe | | | | | |
| 53 | SevSneexe | Severity of sneezing | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 54 | SevitchPalate | Severity of itching of palate | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 55 | SevWatrEye | Severity of watery eyes | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 56 | SevitchEye | Severity of itchy eyes | 0 = no symptoms | * | | | | |
| | | | 1 = very mild | | | | | |
| | | | 2 = mild | | | | | |
| | | | 3 = moderate | | | | | |
| | | | 4 = severe | | | | | |
| 57 | SevSoreEye | Severity of sore eyes | 0 = no symptoms | * | * | | | |
| | | | 1 = very mild | | | | | |
| | | | 2 = mild | | | | | |
| | | | 3 = moderate | | | | | |
| | | | 4 = severe | | | | | |
| 58 | SevSwollEye | Severity of swollen eyes | 0 = no symptoms | * | * | | | |
| | | | 1 = very mild | | | | | |
| | | | 2 = mild | | | | | |
| | | | 3 = moderate | | | | | |
| | | | 4 = severe | | | | | |
| 59 | NumYrsSymp | Number of years symptomatic | — | * | | | | |
| 60 | SympJan | Symptoms occur in January | 0 = absent | * | | | | |
| | | | 1 = present | | | | | |
| | | | 2 = present + | | | | | |
| | | | severe | | | | | |
| 61 | SympFeb | Symptoms occur in February | 0 = absent | * | | | | |
| | | | 1 = present | | | | | |
| | | | 2 = present + | | | | | |
| | | | severe | | | | | |
| 62 | SympMar | Symptoms occur in March | 0 = absent | * | | | | |
| | | | 1 = present | | | | | |
| | | | 2 = present + | | | | | |
| | | | severe | | | | | |
| 63 | SympApr | Symptoms occur in April | 0 = absent | * | | | | |
| | | | 1 = present | | | | | |
| | | | 2 = present + | | | | | |
| | | | severe | | | | | |
| 64 | SympMay | Symptoms occur in May | 0 = absent | * | | | | |
| | | | 1 = present | | | | | |
| | | | 2 = present + | | | | | |
| | | | severe | | | | | |
| 65 | SympJun | Symptoms occur in June | 0 = absent | * | | | | |
| | | | 1 = present | | | | | |
| | | | 2 = present + | | | | | |
| | | | severe | | | | | |
| 66 | SympJul | Symptoms occur in July | 0 = absent | * | | | | |
| | | | 1 = present | | | | | |
| | | | 2 = present + | | | | | |
| | | | severe | | | | | |
| 67 | SympAug | Symptoms occur in August | 0 = absent | * | | | | |
| | | | 1 = present | | | | | |
| | | | 2 = present + | | | | | |
| | | | severe | | | | | |
| 68 | SympSep | Symptoms occur in September | 0 = absent | * | | | | |
| | | | 1 = present | | | | | |
| | | | 2 = present + | | | | | |
| | | | severe | | | | | |
| 69 | SympOct | Symptoms occur in October | 0 = absent | * | | | | |
| | | | 1 = present | | | | | |
| | | | 2 = present + | | | | | |
| | | | severe | | | | | |
| 70 | SympNov | Symptoms occur in November | 0 = absent | * | | | | |
| | | | 1 = present | | | | | |
| | | | 2 = present + | | | | | |
| | | | severe | | | | | |
| 71 | SympDec | Symptoms occur in December | 0 = absent | * | | | | |
| | | | 1 = present | | | | | |
| | | | 2 = present + | | | | | |
| | | | severe | | | | | |
| 72 | BetHome | Symptoms better at home | 0 = no | * | | | | |
| | | | 1 = yes | | | | | |
| 73 | BetWork | Symptoms better at work | 0 = no | * | | | | |
| | | | 1 = yes | | | | | |
| 74 | BetOverseas | Symptoms better overseas | 0 = no | * | * | | | |
| | | | 1 = yes | | | | | |
| 75 | WrseMorn | Symptoms worse in the morning | 0 = no | * | * | * | * | |
| | | | 1 = yes | | | | | |
| 76 | WrseEve | Symptoms worse in the evening | 0 = no | * | | | | |
| | | | 1 = yes | | | | | |
| 77 | WrseDay | Symptoms worse throughout the day | 0 = no | * | | | | |
| | | | 1 = yes | | | | | |
| 78 | WrseNight | Symptoms worse at night | 0 = no | * | | | | |
| | | | 1 = yes | | | | | |
| 79 | AwakeWithSymp | Awake with symptoms | 0 = no | * | | | | |
| | | | 1 = yes | | | | | |
| 80 | NumDaysSympWeek | Number of days symptomatic per week | — | * | | | | |
| 81 | NumHrsSympDay | Number of hours symptomatic per day | — | * | * | * | | |
| 82 | WrseIndoors | Symptoms worse indoors | 0 = no | * | | | | |
| | | | 1 = yes | | | | | |
| 83 | WrseOutdoors | Symptoms worse outdoors | 0 = no | * | | | | |
| | | | 1 = yes | | | | | |
| 84 | SameIn + Out | Symptoms the same indoors and outdoors | 0 = no | * | * | | | |
| | | | 1 = yes | | | | | |
| 87 | WrseDusting | Symptoms worse dusting | 0 = no | * | * | | | |
| | | | 1 = yes | | | | | |
| 86 | WrseGardening | Symptoms worse gardening | 0 = no | * | | | | |
| | | | 1 = yes | | | | | |
| 87 | WrseVacuuming | Symptoms worse vacuuming/cleaning | 0 = no | * | * | * | * | * |
| | | | 1 = yes | | | | | |
| 88 | WrseTraffic | Symptoms worse sitting in traffic jams | 0 = no | * | | | | |
| | | | 1 = yes | | | | | |
| 89 | TriedAviod | Tried avoiding anything | 0 = no | * | * | | | |
| | | | 1 = yes | | | | | |
| 90 | AviodEffective | Avoidance provides symptomatic relief | 0 = no | * | * | | | |
| | | | 1 = yes | | | | | |
| 91 | ContCat | Regular contact with cats | 0 = no | * | | | | |
| | | | 1 = yes | | | | | |
| 92 | WrseCat | Cats make symptoms worse | 0 = no | * | | | | |
| | | | 1 = yes | | | | | |
| 93 | ContDog | Regular contact with dogs | 0 = no | * | | | | |
| | | | 1 = yes | | | | | |
| 94 | WrseDog | Dogs make symptoms worse | 0 = no | * | | | | |
| | | | 1 = yes | | | | | |
| 95 | ContDust | Regular contact with dusts | 0 = no | * | | | | |
| | | | 1 = yes | | | | | |
| 96 | WrseDust | Dusts make symptoms worse | 0 = no | * | | | | |
| | | | 1 = yes | | | | | |
| 97 | ContChem | Regular contact with chemicals | 0 = no | * | | | | |
| | | | 1 = yes | | | | | |
| 98 | WrseChem | Chemicals make symptoms worse | 0 = no | * | | | | |
| | | | 1 = yes | | | | | |
| 99 | HomeDGlaz | Home double glazed | 0 = no | * | | | | |
| | | | 1 = yes | | | | | |
| 100 | HomeCHeat | Home centrally heated | 0 = no | * | | | | |
| | | | 1 = yes | | | | | |
| 101 | HomeFittCarp | Fitted carpets in home | 0 = no | * | | | | |
| | | | 1 = yes | | | | | |
| 102 | HomeDampPat | Damp patches in home | 0 = no | * | * | | | |
| | | | 1 = yes | | | | | |
| 103 | HomeMouldPat | Mould patches in home | 0 = no | * | | | | |
| | | | 1 = yes | | | | | |
| 104 | PiritMths | Months therapeutic trial with Piriton | — | * | | | | |
| 105 | PiritEffect | Therapeutic trial with Piriton effective | 0 = never trialled | * | * | * | * | |
| | | | 1 = no | | | | | |
| | | | 2 = yes | | | | | |
| 106 | PiritStill | Still take Piriton | 0 = never trialled | * | | | | |
| 107 | ZirtMths | Months therapeutic trial with Zirtek | — | * | | | | |
| 10 | ZirtEffect | Therapeutic trial with Zirtek effective | 0 = never trialled | * | | | | |
| | | | 1 = no | | | | | |
| | | | 2 = yes | | | | | |
| 109 | ZirtStill | Still take Zirtek | 0 = never trialled | * | | | | |
| | | | 1 = no | | | | | |
| | | | 2 = yes | | | | | |
| 110 | ClaritMths | Months therapeutic trial with Clarityn | — | * | | | | |
| 111 | ClaritEffect | Therapeutic trial with Clarityn effective | 0 = never trialled | * | | | | |
| | | | 1 = no | | | | | |
| | | | 2 = yes | | | | | |
| 112 | ClaritStill | Still take Clarityn | 0 = never trialled | * | | | | |
| | | | 1 = no | | | | | |
| | | | 2 = yes | | | | | |
| 113 | TriludMths | Months therapeutic trial with Triludan | — | * | | | | |
| 114 | TriludEffect | Therapeutic trial with Triludan effective | 0 = never trialled | * | | | | |
| | | | 1 = no | | | | | |
| | | | 2 = yes | | | | | |
| 115 | TriludStill | Still take Triludan | 0 = never trialled | * | | | | |
| | | | 1 = no | | | | | |
| | | | 2 = yes | | | | | |
| 116 | HismanMths | Months therapeutic trial with Hismanal | — | * | | | | |
| 117 | HismanEffect | Therapeutic trial with Hismanal effective | 0 = never trialled | * | | | | |
| | | | 1 = no | | | | | |
| | | | 2 = yes | | | | | |
| 118 | HismanStill | Still take Hismanal | 0 = never trialled | * | | | | |
| | | | 1 = no | | | | | |
| | | | 2 = yes | | | | | |
| 119 | TelfMths | Months therapeutic trial with Telfast | — | * | | | | |
| 120 | TelfEffect | Therapeutic trial with Telfast effective | 0 = never trialled | * | | | | |
| | | | 1 = no | | | | | |
| | | | 2 = yes | | | | | |
| 121 | TelfStill | Still take Telfast | 0 = never trialled | * | | | | |
| | | | 1 = no | | | | | |
| | | | 2 = yes | | | | | |
| 122 | BeconMths | Months therapeutic trial with Beconase | — | * | | | | |
| 123 | BeconEffect | Therapeutic trial with Beconase effective | 0 = never trialled | * | | | | |
| | | | 1 = no | | | | | |
| | | | 2 = yes | | | | | |
| 124 | BeconStill | Still take Beconase | 0 = never trialled | * | * | * | * | * |
| | | | 1 = no | | | | | |
| | | | 2 = yes | | | | | |
| 125 | FlixMths | Months therapeutic trial with Flixonase | — | * | | | | |
| 126 | FlixEffect | Therapeutic trial with Flixonase effective | 0 = never trialled | * | | | | |
| | | | 1 = no | | | | | |
| | | | 2 = yes | | | | | |
| 127 | FlixStill | Still take Flixonase | 0 = never trialled | * | | | | |
| | | | 1 = no | | | | | |
| | | | 2 = yes | | | | | |
| 128 | RhinoMths | Months therapeutic trial with Rhinocort | — | * | | | | |
| 129 | RhinoEffect | Therapeutic trial with Rhinocort effective | 0 = never trialled | * | | | | |
| | | | 1 = no | | | | | |
| | | | 2 = yes | | | | | |
| 130 | RhinoStill | Still take Rhinocort | 0 = never trialled | * | * | * | * | * |
| | | | 1 = no | | | | | |
| | | | 2 = yes | | | | | |
| 131 | NasoMths | Months therapeutic trial with Nasonex | — | * | | | | |
| 132 | NasoEffect | Therapeutic trial with Nasonex effective | 0 = never trialled | * | * | * | * | |
| | | | 1 = no | | | | | |
| | | | 2 = yes | | | | | |
| 133 | NasoStill | Still take Nasonex | 0 = never trialled | * | * | | | |
| | | | 1 = no | | | | | |
| | | | 2 = yes | | | | | |
| 134 | NasactMths | Months therapeutic trial with Nasacort | — | * | | | | |
| 135 | NasactEffect | Therapeutic trial with Nasacort effective | 0 = never trialled | * | | | | |
| | | | 1 = no | | | | | |
| | | | 2 = yes | | | | | |
| 136 | NasactStill | Still take Nasacort | 0 = never trialled | * | * | | | |
| | | | 1 = no | | | | | |
| | | | 2 = yes | | | | | |
| 137 | RapOptMths | Months therapeutic trial with Rapitil/Opticrom | — | * | | | | |
| 138 | RapOptEffect | Therapeutic trial with Rapitil/Opticrom | 0 = never trialled | * | | | | |
| | | effective | 1 = no | | | | | |
| | | | 2 = yes | | | | | |
| 139 | RapOptStill | Still take Rapitil/Opticrom | 0 = never trialled | * | | | | |
| | | | 1 = no | | | | | |
| | | | 2 = yes | | | | | |
| 140 | SudaMths | Months therapeutic trial with Sudafed | — | * | | | | |
| 141 | SudaEffect | Therapeutic trial with Sudafed effective | 0 = never trialled | * | * | * | * | * |
| | | | 1 = no | | | | | |
| | | | 2 = yes | | | | | |
| 142 | SudaStill | Still take Sudafed | 0 = never trialled | * | | | | |
| | | | 1 = no | | | | | |
| | | | 2 = yes | | | | | |
| 143 | OtrivMths | Months therapeutic trial with Otrivine | — | * | | | | |
| 144 | OrtivEffect | Therapeutic trial with Otrivine effective | 0 = never trialled | * | * | * | * | * |
| | | | 1 = no | | | | | |
| | | | 2 = yes | | | | | |
| 145 | OtrivStill | Still take Otrivine | 0 = never trialled | * | * | | | |
| | | | 1 = no | | | | | |
| | | | 2 = yes | | | | | |
| 146 | Wheeze | Have persistent wheeze | 0 = no | * | | | | |
| | | | 1 = yes | | | | | |
| 147 | Cough | Have persistent cough | 0 = no | * | | | | |
| | | | 1 = yes | | | | | |
| 148 | ChestTight | Have persistent chest tightness | 0 = no | * | | | | |
| | | | 1 = yes | | | | | |
| 149 | DiagAsthma | Diagnosed with bronchial asthma | 0 = no | * | | | | |
| | | | 1 = yes | | | | | |
| 150 | InhalerUse | Use of inhaler(s) for chest | 0 = not applicable | * | | | | |
| | | | 1 = prn | | | | | |
| | | | 2 = regular | | | | | |
| 151 | VentYrs | Years therapeutic trial with Ventolin | — | * | | | | |
| 152 | VentEffect | Therapeutic trial with Ventolin effective | 0 = never trialled | * | | | | |
| | | | 1 = no | | | | | |
| | | | 2 = yes | | | | | |
| 153 | VentStill | Still take Ventolin | 0 = never trialled | * | | | | |
| | | | 1 = no | | | | | |
| | | | 2 = yes | | | | | |
| 154 | SerevYrs | Years therapeutic trial with Serevent | — | * | | | | |
| 155 | SerevEffect | Therapeutic trial with Serevent effective | 0 = never trialled | * | | | | |
| | | | 1 = no | | | | | |
| | | | 2 = yes | | | | | |
| 156 | SerevStill | Still take Serevent | 0 = never trialled | * | | | | |
| | | | 1 = no | | | | | |
| | | | 2 = yes | | | | | |
| 157 | BecotYrs | Years therapeutic trial with Becotide | — | * | | | | |
| 158 | BecotEffect | Therapeutic trial with Becotide effective | 0 = never trialled | * | | | | |
| | | | 1 = no | | | | | |
| | | | 2 = yes | | | | | |
| 159 | BecotStill | Still take Becotide | 0 = never trialled | * | | | | |
| | | | 1 = no | | | | | |
| | | | 2 = yes | | | | | |
| 160 | BecloYrs | Years therapeutic trial with Beclofort | — | * | | | | |
| 161 | BecloEffect | Therapeutic trial with Beclofort effective | 0 = never trialled | * | | | | |
| | | | 1 = no | | | | | |
| | | | 2 = yes | | | | | |
| 162 | BecloStill | Still take Beclofort | 0 = never trialled | * | | | | |
| | | | 1 = no | | | | | |
| | | | 2 = yes | | | | | |
| 163 | FlixoYrs | Years therapeutic trial with Flixotide | — | * | | | | |
| 164 | FlixoEffect | Therapeutic trial with Flixotide effective | 0 = never trialled | * | | | | |
| | | | 1 = no | | | | | |
| | | | 2 = yes | | | | | |
| 165 | FlixoStill | Still take Flixotide | 0 = never trialled | * | | | | |
| | | | 1 = no | | | | | |
| | | | 2 = yes | | | | | |
| 166 | SeretYrs | Years therapeutic trial with Seretide | — | * | | | | |
| 167 | SeretEffect | Therapeutic trial with Seretide effective | 0 = never trialled | * | | | | |
| | | | 1 = no | | | | | |
| | | | 2 = yes | | | | | |
| 168 | SeretStill | Still take Seretide | 0 = never trialled | * | | | | |
| | | | 1 = no | | | | | |
| | | | 2 = yes | | | | | |
| 169 | GrSptHDM | Graded skin prick test result to house dust | 0 = negative | * | * | * | * | * |
| | | mite | 1 = <histamine | | | | | |
| | | | 2 = ≧histamine | | | | | |
| 170 | GrSptCat | Graded skin prick test result to cat | 0 = negative | * | | | | |
| | | | 1 = <histamine | | | | | |
| | | | 2 = ≧histamine | | | | | |
| 171 | GrSptDog | Graded skin prick test result to dog | 0 = negative | * | | | | |
| | | | 1 = <histamine | | | | | |
| | | | 2 = ≧histamine | | | | | |
| 172 | GrSptMGP | Graded skin prick test result to mixed grass | 0 = negative | * | * | * | * | * |
| | | pollens | 1 = <histamine | | | | | |
| | | | 2 = ≧histamine | | | | | |
| 173 | GrSptMTP | Graded skin prick test result to mixed tree | 0 = negative | * | | | | |
| | | pollens | 1 = <histamine | | | | | |
| | | | 2 = ≧histamine | | | | | |
| 174 | GrSptEgg | Graded skin prick test result to egg | 0 = negative | * | * | * | | |
| | | | 1 = <histamine | | | | | |
| | | | 2 = ≧histamine | | | | | |
| 175 | GrSptMilk | Graded skin prick test result to milk | 0 = negative | * | * | * | | |
| | | | 1 = <histamine | | | | | |
| | | | 2 = ≧histamine | | | | | |
| 176 | GrSptRice | Graded skin prick test result to rice | 0 = negative | * | * | | | |
| | | | 1 = <histamine | | | | | |
| | | | 2 = ≧histamine | | | | | |
| 177 | GrSptPeanut | Graded skin prick test result to peanut | 0 = negative | * | | | | |
| | | | 1 = <histamine | | | | | |
| | | | 2 = ≧histamine | | | | | |
| 178 | GrSptHzInut | Graded skin prick test result to hazeInut | 0 = negative | * | | | | |
| | | | 1 = <histamine | | | | | |
| | | | 2 = ≧histamine | | | | | |
| 179 | MagIgE | Graded result of total IgE concentration | 0 = 0-40 | * | * | | | |
| | | | 1 = 41-80 | | | | | |
| | | | 2 = 81-200 | | | | | |
| | | | 3 = 201-1000 | | | | | |
| 180 | GrRstHDM | Graded RAST test result to house dust mite | 0 = not undertaken | * | * | * | * | * |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 181 | GrRstCat | Graded RAST test result to cat | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 182 | GrRstDog | Graded RAST test result to dog | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 183 | GrRstMGP | Graded RAST test result to mixed grass | 0 = not undertaken | * | * | * | * | * |
| | | pollens | 1 = negative | | | | | |
| | | | 2 = mild (1, 2) | | | | | |
| | | | 3 = moderate (3, 4) | | | | | |
| | | | 4 = severe (5, 6) | | | | | |
| 184 | GrRstMTP | Graded RAST test result to mixed tree | 0 = not undertaken | * | | | | |
| | | pollens | 1 = negative | | | | | |
| | | | 2 = mild (1, 2) | | | | | |
| | | | 3 = moderate (3, 4) | | | | | |
| | | | 4 = severe (5, 6) | | | | | |
| 185 | GrRstMilk | Graded RAST test result to milk | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 186 | GrRstHzInut | Graded RAST test result to hazeInut | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 187 | GrRstHDst | Graded RAST test result to house dust | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 188 | GrRstDFar | Graded RAST test result to D. Farinae | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |
| 189 | GrRstMxMould | Graded RAST test result to mixed moulds | 0 = not undertaken | * | | | | |
| | | | 1 = negative | | | | | |
| | | | 2 = mild (1, 2) | | | | | |
| | | | 3 = moderate (3, 4) | | | | | |
| | | | 4 = severe (5, 6) | | | | | |

TABLE 8

| No | Input question | Normalised values |
|---|---|---|
| 1 | Patent gender | 1 = male 2 = female |
| 2 | Patient age in years | — |
| 3 | Do you have asthma or eczema | 0 = no 1 = yes |
| 4 | Taking thyroxine for thyroid disease | 0 = no 1 = yes |
| 5 | Taking any drugs known to activate mast cells | 0 = no 1 = yes |
| 6 | Number of first degree relatives with asthma, | 0 = none X = No specified |
| | eczema or rhinitis | |
| 7 | Severity of runny nose | 0 = no symptoms 1 = very mild 2 = mild 3 = moderate 4 = severe |
| 8 | Severity of itchy nose | 0 = no symptoms 1 = very mild 2 = mild 3 = moderate 4 = severe |
| 9 | Severity of blocked nose | 0 = no symptoms 1 = very mild 2 = mild 3 = moderate 4 = severe |
| 10 | Severity of post nasal discharge | 0 = no symptoms 1 = very mild 2 = mild 3 = moderate 4 = severe |
| 11 | Severity of sneezing | 0 = no symptoms 1 = very mild 2 = mild 3 = moderate 4 = severe |
| 12 | Severity of itching of palate | 0 = no symptoms 1 = very mild 2 = mild 3 = moderate 4 = severe |
| 13 | Severity of watery eyes | 0 = no symptoms 1 = very mild 2 = mild 3 = moderate 4 = severe |
| 14 | Severity of itchy eyes | 0 = no symptoms 1 = very mild 2 = mild 3 = moderate 4 = severe |
| 15 | Severity of sore eyes | 0 = no symptoms 1 = very mild 2 = mild 3 = moderate 4 = severe |
| 16 | Severity of swollen eyes | 0 = no symptoms 1 = very mild 2 = mild 3 = moderate 4 = severe |
| 17 | Symptoms perennial/worse in winter months | 0 = no 1 = yes |
| 18 | Symptoms worse in summer months | 0 = no 1 = yes |
| 19 | Symptoms better at home | 0 = no 1 = yes |
| 20 | Symptoms better at work | 0 = no 1 = yes |
| 21 | Symptoms in the morning, at night, or waking | 0 = no 1 = yes |
| | with symptoms | |
| 22 | Symptoms worse indoors | 0 = no 1 = yes |
| 23 | Symptoms worse outdoors | 0 = no 1 = yes |
| 24 | Symptoms worse dusting and/or vacuuming/cleaning | 0 = no 1 = yes |
| 25 | Symptoms worse gardening | 0 = no 1 = yes |
| 26 | Effective therapeutic trial with antihistamine | 0 = no 1 = yes |
| | or topical nasal steroid | |
| 27 | Graded skin prick test result to house dust mite | 0 = negative 1 = <histamine 2 == histamine |
| 28 | Graded skin prick test result to cat | 0 = negative 1 = <histamine 2 == histamine |
| 29 | Graded skin prick test result to dog | 0 = negative 1 = <histamine 2 == histamine |
| 30 | Graded skin prick test result to mixed grass pollens | 0 = negative 1 = <histamine 2 == histamine |
| 31 | Graded skin prick test result to mixed tree pollens | 0 = negative 1 = <histamine 2 == histamine |
| 32 | Graded skin prick test result to egg | 0 = negative 1 = <histamine 2 == histamine |
| 33 | Graded skin prick test result to milk | 0 = negative 1 = <histamine 2 == histamine |
| 34 | Graded skin prick test result to rice | 0 = negative 1 = <histamine 2 == histamine |
| 35 | Graded skin prick test result to peanut | 0 = negative 1 = <histamine 2 == histamine |
| 36 | Graded skin prick test result to hazeInut | 0 = negative 1 = <histamine 2 == histamine |
| 37 | Graded result of total IgE concentration | 0 = 0-40 1 = 41-80 2 = 81-200 3 = 201-1000 |
| 38 | RAST test result to house dust mite | 0 = neg 1, 2 = mild 3, 4 = moderate 5, 6 = severe |
| 39 | RAST test result to cat | 0 = neg 1, 2 = mild 3, 4 = moderate 5, 6 = severe |
| 40 | RAST test result to dog | 0 = neg 1, 2 = mild 3, 4 = moderate 5, 6 = severe |
| 41 | RAST test result to mixed grass pollens | 0 = neg 1, 2 = mild 3, 4 = moderate 5, 6 = severe |
| 42 | RAST test result to mixed tree pollens | 0 = neg 1, 2 = mild 3, 4 = moderate 5, 6 = severe |
| 43 | RAST test result to milk | 0 = neg 1, 2 = mild 3, 4 = moderate 5, 6 = severe |
| 44 | RAST test result to mixed moulds | 0 = neg 1, 2 = mild 3, 4 = moderate 5, 6 = severe |
| 45 | Symptoms after dietary salicylates | 0 = no 1 = yes |
| 46 | Symptoms after contact with cats, dogs, birds or rodents | 0 = no 1 = yes |
| 47 | How many years have symptoms been present for | X = No specified |

Models Input utilised in (*):

| No | 47 | 23 | 21 | 19 | 15 | 14 | 12 | 9 |
|---|---|---|---|---|---|---|---|---|
| 1 | * | | | | | | | |
| 2 | * | * | * | | * | | | |
| 3 | * | * | * | * | | | | |
| 4 | * | | | | | | | |
| 5 | * | * | * | * | * | * | * | * |
| 6 | * | * | * | * | | | | |
| 7 | * | *$ | *$ | *$ | *$ | *$ | *$ | *$ |
| 8 | * | | | | | | | |
| 9 | * | | | | | | | |
| 10 | * | | | | | | | |
| 11 | * | | | | | | | |
| 12 | * | | | | | | | |
| 13 | * | *$$ | *$$ | *$$ | *$$ | *$$ | | |
| 14 | * | | | | | | | |
| 15 | * | | | | | | | |
| 17 | * | * | * | * | * | * | * | * |
| 18 | * | * | | | | | | |
| 19 | * | | | | | | | |
| 20 | * | * | | | | | | |
| 21 | * | | | | | | | |
| 22 | * | * | * | * | | | * | |
| 23 | * | | | | | | | |
| 24 | * | * | * | * | * | * | * | * |
| 25 | * | * | * | * | * | * | * | |
| 26 | * | * | * | * | | | | |
| 27 | * | * | * | * | * | * | * | * |
| 28 | * | * | * | * | * | * | | |
| 29 | * | | | | | | | |
| 30 | * | * | * | * | * | * | * | * |
| 31 | * | | | | | | | |
| 32 | * | | | | | | | |
| 33 | * | | | | | | | |
| 34 | * | | | | | | | |
| 35 | * | | | | | | | |
| 36 | * | | | | | | | |
| 37 | * | * | * | * | * | * | | |
| 38 | * | * | * | * | * | * | * | * |
| 39 | * | * | * | * | * | * | * | |
| 40 | * | | | | | | | |
| 41 | * | * | * | * | * | * | * | * |
| 42 | * | * | * | * | | | | |
| 43 | * | | | | | | | |
| 44 | * | | | | | | | |
| 45 | * | * | * | * | * | * | * | * |
| 46 | * | | | | | | | |
| 47 | * | * | * | | | | | |

$ = severity of nasal symptoms
$$ = severity of eye symptoms

The following Table, Table 9, summarises the number of patients who were correctly classified as having any form of rhinitis (columns 2 and 3) or allergic perennial rhinitis (columns 4 and 5) using input models 47, 23, 21, 19, 15, 14, 12 or 9 (column 1).

TABLE 9
% correctly classified with 47, 23, 21, 19, 15, 14, 12 and inputs

| | % of total, | % of total, | % of allergic | % of allergic |
|---|---|---|---|---|
| No of | neural | genetic | perennial, | perennial, |
| inputs | analysis | analysis | neural analysis | genetic analysis |
| 47 | 53.3 | 64.3 | 75 | 93 |
| 23 | 89.7 | 82.8 | 93 | 100 |
| 21 | 93 | 82.8 | 100 | 93 |
| 19 | 89.7 | 79.3 | 93 | 100 |
| 15 | 86.3 | 70 | 93 | 93 |
| 14 | 82.8 | 70 | 93 | 93 |
| 12 | 82.8 | 75.9 | 93 | 93 |
| 9 | 82.8 | 75.9 | 93 | 93 |

With this information we were able to determine that the input models we had selected provided a satisfactory level of diagnosis.

Moreover, it could be seen that the predictive value of the data using the 9-input model was as good as the 12-input model. However, when we examined the relative importance of each input in each model we found that only 6 of the 9 inputs in the 9-input model had an appreciable influence in determining the ANN categorisation (Table 10) whereas 11 of the 12 inputs in the 12-input model had such an influence (Table 11).

TABLE 10
Importance of 9 inputs

| | | |
|---|---|---|
| 0.304 | RAST grade house dust mite | |
| 0.283 | Graded SPT result to house dust mite | 0 = neg 1 = <hist 2 = >hist |
| 0.143 | RAST grade grass pollens | |
| 0.103 | Symptoms after dietary salicylate | 0 = No 1 = Yes |
| 0.097 | Taking Mast Cell Activating drugs | 0 = No 1 = Yes |
| 0.069 | Are nasal symptoms perennial or worse in winter | 0 = No 1 = Yes |
| 0.001 | Graded SPT result to grass pollens | 0 = Neg 1 = <hist 2 = >hist |
| 0.000 | Are symptoms worse after dusting or hovering | 0 = No 1 = Yes |
| 0.000 | Severity of nasal symptoms | 0 = none 1 = very mild 2 = mild 3 = moderate 4 = severe |

TABLE 11
Importance of 12 inputs

| | | |
|---|---|---|
| 0.149 | Graded SPT result to house dust mite | 0 = neg 1 = <hist 2 = >hist |
| 0.138 | RAST grade cat | |
| 0.134 | Graded SPT result grass pollens | 0 = neg 1 = <hist 2 = >hist |
| 0.101 | Taking Mast Cell Activating drugs | 0 = No 1 = Yes |
| 0.100 | Severity of nasal symptoms | 0 = none 1 = very mild 2 = mild 3 = moderate 4 = severe |
| 0.093 | Are symptoms worse after dusting or hovering | 0 = No 1 = Yes |
| 0.084 | Graded SPT result to grass pollens | 0 = Neg 1 = <hist 2 = >hist |
| 0.064 | Are symptoms worse indoors | 0 = No 1 = Yes |
| 0.051 | Are symptoms worse after gardening | 0 = No 1 = Yes |
| 0.043 | Are nasal symptoms perennial or worse in winter | 0 = No 1 = Yes |
| 0.039 | Symptoms after dietary salicylate | 0 = No 1 = Yes |
| 0.005 | RAST grade house dust mite | |

Moreover, we were also mindful of the fact that an input model with too few data fields might skew the classification and so for this reason the 12-input model is our favoured model because it seems to strike a balance between decision making with as few input data fields as reasonably possible whilst not missing the possible important influence of the extra input data fields in determining categorisation when a large number of patients are analysed using the ANN.

### Statistics

The performance of the optimal model on bootstrap test data and blind validation data was assessed using Receiver Operating Characteristic (ROC) curves. These curves provided information on the predictive accuracy, sensitivity, specificity, positive predictive value and negative predictive value for output diagnoses. The area under the curve (AUC) was calculated as a measure of discrimination.

### Results

### Patient Characteristics

During the data collection period 6 Oct. 2003, to 29 Jan. 2004, 93 patients referred to the WCAS with symptoms of rhinitis attended outpatient clinics and consented to participation in the study. Two patients in whom final diagnoses of infective sinusitis were made were excluded from the study; the remaining 91 patients (31 [34.1%] men, 60 [65.9%] women; mean age 41.3 years [SD 15.6]) were included. Consultant Clinical Immunologist-derived data from patients 001-062 (n=62) was used to train the ANN. Data from the remaining 29 patients was utilised for blind validation of the optimal ANN model produced following parameterisation. The training and validation groups were similar in terms of demographic features and distribution of output diagnoses (Table 12).

TABLE 12
Characteristics of patients presenting
to the WCAS with symptoms of rhinitis

| | Training and test | Validation |
|---|---|---|
| | subset (6 Oct. 2003- | subset (15 Jan. 2004- |
| Characteristic | 12 Jan. 2004) (n = 62) | 29 Jan. 2004) (n = 29) |
| Mean age in years (SD) | 43.1 (15.7) | 37.5 (14.9) |
| Male/Female | 21 (33.9%)/41 (66.1%) | 10 (34.5%)/19 (65.5%) |
| Output diagnosis | 43 (69%) | 16 (55.2%) |
| allergic perennial | | |
| Output diagnosis | 7 (11%) | 8 (27.7%) |
| allergic seasonal | | |
| Output diagnosis | 3 (5%) | 3 (10.3%) |
| idiopathic perennial | | |
| Output diagnosis | 2 (3%) | 1 (3.4%) |
| idiopathic seasonal | | |
| Output diagnosis | 5 (8%) | 1 (3.4%) |
| drug induced | | |
| Output diagnosis | 2 (3%) | 0 (0%) |
| dietary salicylate | | |

### CONCLUSION

This study has provided evidence that data collected by structured questionnaire and analysed by ANN software can correctly diagnose upper respiratory tract disorders, such as rhinitis, by aetiological cause.

### REFERENCES

Basheer, I, A and Hajmeer, M. (2000). ‘Artificial neural networks: fundamentals, computing, design and application’. J. Microbiol Methods 43: 3-31.

Baxt, W, G. (1995). ‘Application of Artificial Neural Networks to Clinical Medicine.’ Lancet 346: 1135-1138.

Callan, R. (1999). ‘The Essence of Neural Networks’. Hemel Hempstead: Prentice Hall Europe.

Das, A; Ben-Menachem, T; Cooper, G, S, et al. (2003). ‘Prediction of outcome in acute lower-gastrointestinal haemorrhage based on an artificial neural network: internal and external validation of a predictive model.’ Lancet 362: 1261-1266.

Dybowski, R and Gant, V (eds) (2001). ‘Clinical Applications of Artificial Neural Networks’. Cambridge: Cambridge University Press.

Hanley, J, A and McNeil, B, J. (1982). ‘The meaning and use of the Area under a Receiver Operating Characteristic (ROC) Curve.’ Radiology 143: 29-36.

Hecht-Nielsen, R. (1990). ‘Neurocomputing’. Massachusetts: Addison-Wesley.

Holgate, S, T. (1999). ‘The epidemiology of allergy and asthma’. Nature 402: (suppl) B2-B4.

Holgate, S, T and Broide, D. (2003). ‘New targets for allergic rhinitis—a disease of civilisation’. Nature Rev Drug Discovery 2: 1-12.

Lancashire, L, J; Mian, S; Rees, R, C, et al. (2003). ‘Preliminary artificial neural network analysis of SELDI mass spectrometry data for the classification of melanoma tissue’. Proceedings of the 17^{th} European Stimulation Multiconference.

Linneberg, A; Nielsen, N, H; Madsen, F, et al. (2000). ‘Increasing prevalence of specific IgE to aeroallergens in an adult population: two cross-sectional surveys 8 years apart: the Copenhagen Allergy Study’. J. Allergy and Clin Imm 106: 247-252.

McCulloh, W, S and Pitts, W. (1943). ‘A logical calculus of the ideas immanent in nervous activity.’ Bull. Math. Biophys 5: 115-133.

Metz, C, E. (1978). ‘Basic principles of ROC analysis.’ Semin. Nuc Med 8: 283-298.

Roadknight, C; Palmer-Brown, D and Al-Dabass, D. (1997). ‘Simulation of correlation activity pruning methods to enhance transparency of ANNs.’ Int. J. Simulation 4: 68-74.

Rosenblatt, F. (1958). ‘The perceptron: a probabilistic model for information storage and organisation in the brain.’ Psychol Rev 65: 386-408.

Rumelhart, D, E and McClelland, J, L. (1986). ‘Parallel distribution processing: Explorations in the microstructure of cognition: Volume 1: Foundations’. Cambridge USA: MIT Press.

Schalkoff, R, J. (1977). ‘Artificial Neural Networks’. New York: McGraw-Hill.

Swingler, K. (1996). ‘Applying Neural Networks: A Practical Guide’. NewYork: Academic Press

Thompson, R. A, Bird A. G (1983). How necessary are specific IgE antibody tests in allergy diagnosis? Lancet, 321, 169-173.

Wei, J, T; Zhang, Z; Barnhill S, D, et al. (1998). ‘Understanding artificial neural networks and exploring their potential applications for the practicing urologist’. Urology 52: 161-172.

Wide, L, Bennich H, Johansson SGO (1967). Diagnosis of allergy by an in vitro test for allergen antibodies. Lancet, 2:1105.

Zweig, M, H and Campbell, G. (1993). ‘Receiver-Operating Characteristic (ROC) plots: a fundamental evaluation tool in clinical medicine.’ Clin. Chem 39: 561-577.

1. A method for diagnosing rhinitis or sinusitis comprising:
(a) asking a patient each of the following questions:
are any drugs being taken that are known to activate MAST cells said drugs selected from the group consisting of alpha blockers, ACE inhibitors/ATII Receptor antagonists, aspirin, 5 HT-1 agonists, opiate or derivative medications, proton pump inhibitors, selective serotonin reuptake inhibitors and statins;
severity of nasal symptoms on a scale of 0-n;
are the symptoms perennial/worse in the winter months;
are the symptoms worse during dusting, vacuuming, or cleaning;
are the symptoms present after dietary salicylates; and
(b) carrying out each of the following tests:
skin prick test to house dust mite and/or cockroach;
skin prick test to mixed pollens;
RAST test result to house dust mite and/or cockroach;
RAST test result to mixed pollens; and
(c) inputting the results of the questions and tests into a neural network trained to diagnose said condition; and
(d) producing an output indicative of a diagnosis.

2. A method according to claim 1 wherein said mixed pollens are selected having regard to the geographical region where the patient resides.

3. A method according to claim 1 wherein said one or more tests involves the provision of a graded result.

4. A method according to claim 1 wherein part (a) thereof further involves asking a patient each of the following questions:
are the symptoms worse indoors;
are the symptoms worse when gardening; and
part (b) thereof further includes carrying out the following further test:
RAST test to cat.

5. A method according to claim 1 wherein part (a) thereof
further involves asking a patient the following questions:
severity of eye symptoms (on a scale of 0-n);
are the symptoms worse when gardening;
and part (b) further includes carrying out the following tests:
RAST test to cat;
skin prick test to cat; and
total IgE concentration.

6. A method according to claim 1 wherein part (a)
thereof further involves asking a patient the following questions:
patient age in years;
severity of eye symptoms;
are symptoms worse when gardening; and
part (b) thereof involves performing the further tests:
skin prick test to cat;
total IgE concentration;
RAST test to cat.

7. A method according to claim 1 wherein part (a)
thereof further involves asking a patient the following questions:
do you have asthma or eczema;
number of first degree relatives with asthma, eczema or rhinitis;
severity of eye symptoms;
symptoms worse indoors;
symptoms worse when gardening;
effective therapeutic trial with antihistamine or topical nasal steroid; and
part (b) thereof involves performing the following further tests:
skin prick test to cat;
total IgE concentration;
RAST test to cat; and
RAST test to mixed pollens.

8. A method according to claim 1 wherein part (a)
thereof involves asking a patient the further questions:
patient age in years;
do you have asthma or eczema;
number of first degree relatives with asthma, eczema or rhinitis;
severity of eye symptoms;
symptoms worse indoors;
symptoms worse when gardening;
effective therapeutic trial with antihistamine or topical nasal steroid;
how many years have symptoms been present; and
part (b) thereof involves performing the further tests:
skin prick test to cat;
total IgE concentration;
RAST test to cat;
RAST test to mixed pollens.

9. A method according to claim 1 wherein part (a)
thereof further involves asking the following questions:
patient age in years;
do you have asthma or eczema;
number of first degree relatives with asthma, eczema or rhinitis;
severity of eye symptoms (0-n);
symptoms worse in summer months;
symptoms better at work;
symptoms worse indoors;
symptoms worse when gardening;
effective therapeutic trial with antihistamine or topical nasal steroid;
for how many years have symptoms been present; and
part (b) thereof comprises performing the following further tests:
skin prick test to cat;
total IgE concentration;
RAST test to cat;
RAST to mixed pollens;
RAST test to mixed grass pollens;
RAST test to mixed tree pollens.

10. A method according to claim 1 which comprises in
part (a) thereof further involves asking a patient all the questions in Table 8; and
part (b) thereof involves performing all the tests in Table 8.

11. A computer system or apparatus, configured to aid in the diagnosis of rhinitis or sinusitis, comprising:
(a) a device for obtaining data relating to a patient, wherein the data comprises answers to any selected combination of questions and results of tests outlined in an input model selected from the group consisting of 9-, 12-, 14-, 15-, 19-, 21-, 23- and 47-input models above;
(b) a device for storing the data in storage means of the computer system;
(c) a device for transferring the data to a neural network trained on samples of the data; and
(d) a device for extracting from the trained neural network an output, the output being an indicator for the diagnosis of rhinitis or sinusitis.

12. A method for training a neural network to aid in diagnosing rhinitis or sinusitis, comprising:
a) obtaining data relating to a group of patients in whom rhinitis or sinusitis is known, wherein the data comprises any selected combination of the results of the questions and tests outlined in an input model selected from the group consisting of 9-, 12-, 14-, 15-, 19-, 21-, 23- and 47-input models;
(b) training a neural network to identify the pattern of data which corresponds to the rhinitis or sinusitis; and
(c) storing the neural network in storage means of a computer or on a computer-readable medium.

13. A computer program product comprising:
a computer usable medium having computer readable program code and computer readable system code embodied on said medium for aiding in the diagnosis of rhinitis or sinusitis, said computer program product including:
computer program code means, when the program code is loaded, to make the computer execute a procedure to:
(a) obtain data relating to a patient, wherein the data comprises answers to any selected combination of questions and results of the tests outlined in an input model selected from the group consisting of 9-, 12-, 14-, 15-, 19-, 21-, 23- and 47-input models;
(b) store the data;
(c) transfer the data to a neural network trained on the aforementioned data; and
(d) extract from the trained neural network an output, the output being an indicator for the diagnosis of rhinitis or sinusitis.

14. A computer system comprising a first means for:
(a) obtaining data relating to a patient, wherein the data comprises answers to any selected combination of questions and results of tests outlined in an input model selected from the group consisting of 9-, 12-, 14-, 15-, 19-, 21-, 23- and 47-input models; and
a second remote means, wherein said second remote means comprises means for:
(b) storing the data;
(c) transferring the data to a neural network trained on the aforementioned data; and
(d) extracting from the trained neural network on output, the output being an indicator for the diagnosis of rhinitis or sinusitis.

15. (canceled)

16. A method according to claim 1 for diagnosing any one of the following conditions:
allergic perennial rhinitis, allergic seasonal rhinitis, idiopathic perennial rhinitis, idiopathic seasonal rhinitis, drug-induced rhinitis, dietary salicylate-induced rhinitis or rhino-sinusitis.

17. (canceled)

18. A computer system or program according to claim 11 wherein the allergy is any one of the following conditions:
allergic perennial rhinitis, allergic seasonal rhinitis, idiopathic perennial rhinitis, idiopathic seasonal rhinitis, drug-induced rhinitis, dietary salicylate-induced rhinitis or rhino-sinusitis.

19-20. (canceled)

21. A computer system or program according to claim 13 wherein the allergy is any one of the following conditions:
allergic perennial rhinitis, allergic seasonal rhinitis, idiopathic perennial rhinitis, idiopathic seasonal rhinitis, drug-induced rhinitis, dietary salicylate-induced rhinitis or rhino-sinusitis.

22. A computer system or program according to claim 14 wherein the allergy is any one of the following conditions:
allergic perennial rhinitis, allergic seasonal rhinitis, idiopathic perennial rhinitis, idiopathic seasonal rhinitis, drug-induced rhinitis, dietary salicylate-induced rhinitis or rhino-sinusitis.
